(19) United States
(12) Patent Application Publication
(10) Pub. No.: US 2025/0087313 A1
(43) Pub. Date: Mar. 13, 2025

(54) SYSTEMS AND METHODS FOR OPTIMIZING A SAMPLE SIZE OF AN INVESTIGATION

(71) Applicant: Optum Services (Ireland) Limited, Dublin (IE)

(72) Inventors: Breanndan O’CONCHUIR, Killarney (IE); Daniel KELLY, Dublin (IE); Conor John WALDRON, Dublin (IE); Michael J. McCARTHY, Dublin (IE)

(21) Appl. No.: 18/463,392

(22) Filed: Sep. 8, 2023

(51) Int. Cl. G16H 10/20

(52) U.S. Cl. CPC G16H 10/20

(57) ABSTRACT

Systems and methods are disclosed for systems and methods for optimizing a sample size of an investigation. A method includes: accessing a plurality of datasets stored in a database; identifying first and second subsets of the plurality of datasets merging the first subset and the second subset into a first data object; receiving, as input via an interactive interface, a user input indicative of a plurality of parameters that correspond to values contained in the first data object; generating a plurality of similarity-based subsets by applying one or more sampling techniques to the first data object across the plurality of parameters, each of the sample similarity-based subsets associated with a deviation measure; generating a second data object comprising one of the plurality of similarity-based subsets associated with a lowest deviation measure; and providing the second data object for display via the interactive interface in response to the user input

### TECHNICAL FIELD

Various embodiments of this disclosure relate generally to techniques for clinical trial cohort generation, and, more particularly, to systems and methods for processing population data by resolving demographic and medical categorizations to output higher quality and more representative clinical trial cohorts.

### BACKGROUND

The current landscape of clinical trials often grapples with the challenge of ensuring that the selected participant cohorts are truly representative of the broader target population. This is a significant issue as the results derived from such trials may be statistically biased and fail to accurately portray the effectiveness of a new treatment or drug across diverse demographic and clinical groups. Traditional approaches to this problem have relied on defining eligibility criteria and employing outreach methods to recruit participants.

However, these methods suffer from one or more issues and may be improved in one or more ways. A key issue lies in the initial estimation of the true prevalence of a condition, especially when a significant proportion of individuals may be undiagnosed or misdiagnosed. This results in an inaccurate picture of the true target population, thereby hindering the effectiveness of devising representative trial quotas.

For instance, current methods may struggle to account for the complexity and diversity of the target population, particularly when there are multiple socioeconomic and medical categories to consider. The task of identifying representative samples across these diverse categories can be daunting, and often, clinical trials fail to meet enrolment timelines due to these complexities.

The consequence of these shortcomings is a clinical trial participant pool that may not adequately represent the broader target population. This could lead to skewed results and an over- or underestimation of the effectiveness of a new treatment or drug on certain demographic or clinical groups. Ultimately, this can impede the advancement of medical research and potentially limit the applicability of new treatments or drugs to all those who may benefit.

Therefore, there is a need for a more sophisticated and accurate approach to cohort generation for clinical trials. This approach should not only account for the complexities and diversities of the target population but also be capable of identifying, comparing, and ranking potential cohorts based on their representativeness across multiple distinct categories. Furthermore, it should also consider those individuals who may be undiagnosed or misdiagnosed, thereby ensuring a more comprehensive and accurate representation of the true target population.

This disclosure is directed to addressing the above-mentioned challenges. The background description provided herein is for the purpose of generally presenting the context of the disclosure. Unless otherwise indicated herein, the materials described in this section are not prior art to the claims in this application and are not admitted to be prior art, or suggestions of the prior art, by inclusion in this section.

### SUMMARY

The present disclosure addresses the technical problem(s) described above or elsewhere in the present disclosure and improves the state of conventional cohort generation techniques, such as those used in the healthcare applications. In some embodiments, the present disclosure teaches systems and methods for cohort generation that accounts for the complexities and diversities of the target population, evaluates potential cohorts based on their representativeness across multiple categories, and ensures a more comprehensive and accurate representation of the target population.

In some aspects, the techniques described herein relate to a computer-implemented method including: accessing, by one or more processors, a plurality of datasets stored in a database; identifying, by the one or more processors, (a) a first subset of the plurality of datasets that each include an indicator explicitly representing one or more conditions based on one or more deterministic criteria, and (b) a second subset of the datasets that excludes the indicator and includes data implicitly representing the one or more conditions based on the one or more deterministic criteria; merging, by the one or more processors, the first subset and the second subset into a first data object; receiving, by the one or more processors and as input via an interactive interface, a user input indicative of a plurality of parameters that correspond to values contained in the first data object; generating, by the one or more processors, a plurality of similarity-based subsets by applying one or more sampling techniques to the first data object across the plurality of parameters, each of the sample similarity-based subsets associated with a deviation measure; generating, by the one or more processors, a second data object including one of the plurality of similarity-based subsets associated with a lowest deviation measure; and providing, by the one or more processors, the second data object for display via the interactive interface in response to the user input.

In some aspects, the techniques described herein relate to a system including: one or more storage devices storing instructions; and one or more processors executing the instructions to perform a process including: accessing a plurality of datasets stored in a database; identifying (a) a first subset of the plurality of datasets that each include an indicator explicitly representing one or more conditions based on one or more deterministic criteria, and (b) a second subset of the datasets that excludes the indicator and includes data implicitly representing the one or more conditions based on the one or more deterministic criteria; merging the first subset and the second subset into a first data object; receiving, as input via an interactive interface, a user input indicative of a plurality of parameters that correspond to values contained in the first data object; generating a plurality of similarity-based subsets by applying one or more sampling techniques to the first data object across the plurality of parameters, each of the sample similarity-based subsets associated with a deviation measure; generating a second data object including one of the plurality of similarity-based subsets associated with a lowest deviation measure; and providing the second data object for display via the interactive interface in response to the user input.

In some aspects, the techniques described herein relate to a non-transitory computer-readable medium storing instructions which, when executed by a computer, cause the computer to perform am method including: accessing, by one or more processors, a plurality of datasets stored in a database; identifying, by the one or more processors, (a) a first subset of the plurality of datasets that each include an indicator explicitly representing one or more conditions based on one or more deterministic criteria, and (b) a second subset of the datasets that excludes the indicator and includes data implicitly representing the one or more conditions based on the one or more deterministic criteria; merging, by the one or more processors, the first subset and the second subset into a first data object; receiving, by the one or more processors and as input via an interactive interface, a user input indicative of a plurality of parameters that correspond to values contained in the first data object; generating, by the one or more processors, a plurality of similarity-based subsets by applying one or more sampling techniques to the first data object across the plurality of parameters, each of the sample similarity-based subsets associated with a deviation measure; generating, by the one or more processors, a second data object including one of the plurality of similarity-based subsets associated with a lowest deviation measure; and providing, by the one or more processors, the second data object for display via the interactive interface in response to the user input.

It is to be understood that both the foregoing general description and the following detailed description are example and explanatory only and are not restrictive of the detailed embodiments, as claimed.

### BRIEF DESCRIPTION OF THE DRAWINGS

The accompanying drawings, which are incorporated in and constitute a part of this specification, illustrate various example embodiments and together with the description, serve to explain the principles of the disclosed embodiments.

FIG. 1A is a diagram showing an example of a system configured for cohort generation, according to some embodiments of the disclosure.

FIG. 1B is a diagram of example components of a cohort generation platform, according to some embodiments of the disclosure.

FIG. 1C is a diagram of example components of a cohort generation module, according to some embodiments of the disclosure.

FIG. 2 is a flowchart showing a method for generating a cohort, according to some embodiments of the disclosure.

FIG. 3A is a flowchart showing one or more steps in a method for generating a cohort, according to some embodiments of the disclosure.

FIG. 3B is a flowchart showing one or more steps in a method for generating a cohort, according to some embodiments of the disclosure.

FIG. 3C is a flowchart showing one or more steps in a method for generating a cohort, according to some embodiments of the disclosure.

FIG. 3D is a flowchart showing one or more steps in a method for generating a cohort, according to some embodiments of the disclosure.

FIG. 4 shows an example machine-learning training flow chart, according to some embodiments of the disclosure.

FIG. 5 illustrates an implementation of a computer system that executes techniques presented herein, according to some embodiments of the disclosure.

### DETAILED DESCRIPTION

Various embodiments of this disclosure relate generally to techniques for clinical trial cohort generation, and, more particularly, to systems and methods for processing population data by resolving demographic and medical categorizations to output higher quality and more representative clinical trial cohorts.

As previously discussed, despite advancements in clinical trial participant selection and recruitment techniques, conventional methods still face certain limitations and challenges. One of these challenges involves adequately understanding the diversity and complexity of the target population, particularly when elements such as race, gender, medical history, comorbidities, and undiagnosed conditions are involved. The intricacies of these multiple categories can often lead to errors and inaccuracies in the selected cohorts. Furthermore, the complex and resource-intensive nature of traditional cohort generation techniques can result in significant computational power and time requirements. Additionally, these traditional approaches often lack the capability to handle variations and anomalies in real-world populations, leading to suboptimal performance and poor representation rates.

In view of the limitations of conventional methodologies, the techniques disclosed herein address these technical issues and aim to substantially enhance the ability to process, understand, and select cohorts from the target population, with particular effectiveness in the context of complex medical studies, for example. By utilizing a unique combination of modules and machine-learning models to identify and standardize categories, detect eligibility, and resolve conflicts, the disclosed system and method improve the accuracy and efficiency of cohort generation. The systems and methods disclosed herein are adapted to effectively consider the intricacies of the target population's diversity and complexity, thereby improving the selection of representative cohorts. The systems and methods disclosed herein are not only capable of managing generalized data sets but also exhibit robust performance with varied and unseen data. By generating cohorts in a variety of formats that are suitable for different use cases in the medical field, the system becomes significantly more versatile, further expanding its applicability and value.

While principles of the present disclosure are described herein with reference to illustrative embodiments for particular applications, it should be understood that the disclosure is not limited thereto. Those having ordinary skill in the art and access to the teachings provided herein will recognize additional modifications, applications, embodiments, and substitution of equivalents all fall within the scope of the embodiments described herein. Accordingly, the invention is not to be considered as limited by the foregoing description.

Various non-limiting embodiments of the present disclosure will now be described to provide an overall understanding of the principles of the structure, function, and use of systems and methods disclosed herein for generating effective cohorts.

Reference to any particular activity is provided in this disclosure only for convenience and not intended to limit the disclosure. A person of ordinary skill in the art would recognize that the concepts underlying the disclosed devices and methods may be utilized in any suitable activity. For example, while the present disclosure is in the context of cohort generation, one of ordinary skill would understand the applicability of the described systems and methods to similar tasks, such as optimizing samples in one or more other investigations. The disclosure may be understood with reference to the following description and the appended drawings, wherein like elements are referred to with the same reference numerals.

The terminology used below may be interpreted in its broadest reasonable manner, even though it is being used in conjunction with a detailed description of certain specific examples of the present disclosure. Indeed, certain terms may even be emphasized below; however, any terminology intended to be interpreted in any restricted manner will be overtly and specifically defined as such in this Detailed Description section. Both the foregoing general description and the following detailed description are exemplary and explanatory only and are not restrictive of the features, as claimed.

In this disclosure, the term “based on” means “based at least in part on.” The singular forms “a,” “an,” and “the” include plural referents unless the context dictates otherwise. The term “exemplary” is used in the sense of “example” rather than “ideal.” The terms “comprises,” “comprising,” “includes,” “including,” or other variations thereof, are intended to cover a non-exclusive inclusion such that a process, method, or product that comprises a list of elements does not necessarily include only those elements, but may include other elements not expressly listed or inherent to such a process, method, article, or apparatus. The term “or” is used disjunctively, such that “at least one of A or B” includes, (A), (B), (A and A), (A and B), etc. Relative terms, such as, “substantially” and “generally,” are used to indicate a possible variation of +10% of a stated or understood value.

It will also be understood that, although the terms first, second, third, etc. are, in some instances, used herein to describe various elements, these elements should not be limited by these terms. These terms are only used to distinguish one element from another. For example, a first contact could be termed a second contact, and, similarly, a second contact could be termed a first contact, without departing from the scope of the various described embodiments. The first contact and the second contact are both contacts, but they are not the same contact.

As used herein, the term “if” is, optionally, construed to mean “when” or “upon” or “in response to determining” or “in response to detecting,” depending on the context. Similarly, the phrase “if it is determined” or “if [a stated condition or event] is detected” is, optionally, construed to mean “upon determining” or “in response to determining” or “upon detecting [the stated condition or event]” or “in response to detecting [the stated condition or event],” depending on the context.

As used herein, a “machine-learning model” generally encompasses instructions, data, and/or a model configured to receive input, and apply one or more of a weight, bias, classification, or analysis on the input to generate an output. The output may include, for example, a classification of the input, an analysis based on the input, a design, process, prediction, or recommendation associated with the input, or any other suitable type of output. A machine-learning model is generally trained using training data, e.g., experiential data and/or samples of input data, which are fed into the model in order to establish, tune, or modify one or more aspects of the model, e.g., the weights, biases, criteria for forming classifications or clusters, or the like. Aspects of a machine-learning model may operate on an input linearly, in parallel, via a network (e.g., a neural network), or via any suitable configuration.

Training the machine-learning model may include one or more machine-learning techniques, such as linear regression, logistical regression, random forest, gradient boosted machine (GBM), deep learning, and/or a deep neural network. Supervised and/or unsupervised training may be employed. For example, supervised learning may include providing training data and labels corresponding to the training data, e.g., as ground truth. Unsupervised approaches may include clustering, classification or the like. K-means clustering or K-Nearest Neighbors may also be used, which may be supervised or unsupervised. Combinations of K-Nearest Neighbors and an unsupervised cluster technique may also be used. Any suitable type of training may be used, e.g., stochastic, gradient boosted, random seeded, recursive, epoch or batch-based, etc. After training the machine-learning mode, the machine-learning model may be deployed in a computer application for use on new input data that it has not been trained on previously.

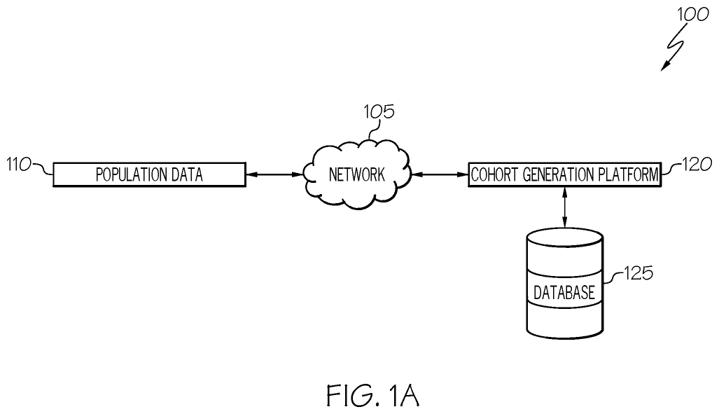

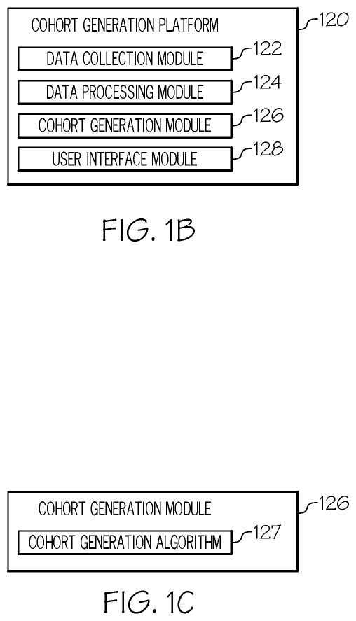

FIG. 1A is a diagram showing an example of a system that is capable of cohort generation, according to some embodiments of the disclosure. The depicted network environment, designated as 100, is in accordance with a specific embodiment of the current invention. The network environment 100 encompasses a communication infrastructure, such as network 105, which is accompanied by population data 110, and is further equipped with a cohort generation platform 120 integrated with a database 125.

In one embodiment, various components of the network environment 100 interact with each other through the network 105. The network 105 facilitates communication between the cohort generation platform 120 and one or more other systems, including one or more datasets, such as (but not limited to) population data 110. The one or more datasets and/or population data 110 includes data and/or one or more data entries associated with or comprising medical records. The network 105 includes one or more networks such as a data network, a wireless network, a telephony network, or any combination thereof.

The population data 110, in some embodiments, consists of structured or unstructured data relating to a population of individuals. The population data 110 can include data such as demographic information, medical records, insurance claims, or other information relevant to a medical trial. The population data 110 is stored in storage, which can be any local or remote data repository such as file servers, cloud-based storage, or other forms of data storage.

The database 125 is used to support the storage and retrieval of data related to one or more datasets, such as the population data 110, storing metadata and/or healthcare data about the population represented in the population data 110, as well as any extracted information from the cohort generation platform 120. The database 125 can consist of one or more systems, such as a relational database management system (RDBMS), a NoSQL database, or a graph database, depending on the requirements and use cases of the network environment 100.

In one embodiment, the database 125 is any type of database, such as relational, hierarchical, object-oriented, etc., wherein data is organized in tables, lookup tables, or other suitable manners. The database 125 stores and provides access to data utilized by the cohort generation platform 120 to identify cohorts. The database 125 stores information related to the population data 110 as well as information generated by the cohort generation platform 120. The database 125 can store various types of information to aid in the cohort generation process.

In one embodiment, the database 125 includes a machine learning-based training database that maps relationships between input parameters from the population data 110 and output parameters representing the generated cohorts. For example, the training database can include machine learning algorithms that learn mappings between demographic or medical data inputs and cohort outputs. The training database can be routinely updated based on additional machine learning.

The cohort generation platform 120 communicates with other components of the network 105 using known or developing protocols. These protocols govern interactions between network nodes and define rules for generating, receiving, and interpreting information sent over communication links. The protocols operate at different layers, from generating physical signals to identifying software applications sending or receiving the information.

Communications between the network nodes are typically effected by exchanging discrete packets of data. Each packet typically comprises (1) header information associated with a particular protocol, and (2) payload information that follows the header information and contains information that may be processed independently of that particular protocol. In some protocols, the packet includes (3) trailer information following the payload and indicating the end of the payload information. The header includes information such as the source of the packet, its destination, the length of the payload, and other properties used by the protocol. Often, the data in the payload for the particular protocol includes a header and payload for a different protocol associated with a different, higher layer of the OSI Reference Model. The header for a particular protocol typically indicates a type for the next protocol contained in its payload. The higher layer protocol is said to be encapsulated in the lower layer protocol. The headers included in a packet traversing multiple heterogeneous networks, such as the Internet, typically include a physical (layer 1) header, a data-link (layer 2) header, an internetwork (layer 3) header and a transport (layer 4) header, and various application (layer 5, layer 6 and layer 7) headers.

In operation, the network environment 100 provides a framework for analyzing large amounts of population data 110, leveraging cohort generation and database technologies to support various use cases and applications. For example, the network environment 100 can be used to generate cohorts from one or more datasets, such as the population data 110, based on user-defined criteria or a plurality of parameters.

To perform these tasks, the cohort generation platform 120 utilizes techniques such as the cohort generation algorithm 127, which analyzes the population data 110 and identifies cohorts matching the specified criteria. The cohort generation platform 120 can also utilize the data collection module 122 and data processing module 124 to gather and prepare the population data 110.

To support storage and retrieval of data related to the generated cohorts, the database 125 stores metadata about the population data 110, such as data sources, types, and formats. The database 125 also stores information about the generated cohorts output by the cohort generation platform 120, such as cohort criteria, identifiers, and statistics.

In addition to cohort generation, the network environment 100 can support other applications like data visualization, search, and predictive modeling. For example, the network environment 100 could allow users to search the population data 110 for individuals matching certain criteria, or visualize cohort statistics through interactive graphs and charts.

FIG. 1B is a diagram of example components of a cohort generation platform, according to some embodiments of the disclosure. Referring to FIG. 1B, the cohort generation platform 120 is a component of the network environment 100. The cohort generation platform 120 provides the capabilities to analyze one or more datasets, such as population data 110 and generate cohorts. As used herein, terms like “component” or “module” encompass hardware and/or software implemented by a processor or the like. For example, the cohort generation platform 120 includes components for collecting, processing, and analyzing population data as well as generating cohorts. The cohort generation platform 120 includes modules such as a data collection module 122, a data processing module 124, a cohort generation module 126, and a user interface module 128. It is contemplated that the functions of these modules could be combined into fewer modules or performed by other modules with equivalent functionality.

In some embodiments, the data collection module 122 of the cohort generation platform 120 undertakes the collection of data from one or more datasets, such as population data 110, during the operation of the environment 100. This involves the reception of protocol data, akin to data from a network 105 associated with the environment 100. The collected data encompasses user interactions, timestamps, items chosen, and other pertinent information associated with the environment 100 or its network 105.

Subsequent to the data collection by data collection module 122, the data processing module 124 of the cohort generation platform 120 partakes in the processing and preparation of the data for further analysis by the cohort generation module 126. The data processing module 124 engages in the cleaning of the data, removal of irrelevant or redundant information, and conversion of the data into a format suitable for further processing by the cohort generation module 126.

The cohort generation module 126, upon receiving the prepared data from data processing module 124, applies algorithms and models, such as cohort generation algorithm 127, to generate a true eligible population and representative categories for the true eligible population, based on the input data. The cohort generation module 126 utilizes various algorithms and employs a variety of models to accomplish its task.

After the cohort generation module 126 has generated the true eligible population and representative categories based on the input data, a user interface generated on a user device via the user interface module 128 displays the results to the user at an appropriate time. The user interface provides an interactive and intuitive interface, enabling the user to view, modify, or confirm the generated results. The user interface also enables the user to provide feedback or additional information to improve the cohort generation process or adjust the cohort generation algorithm 127 accordingly. The user interface module 128 is also configured to receive a user input via an interactive interface, the user input being one or more parameters.

FIG. 1C is a diagram of example components of a cohort generation module, according to some embodiments of the disclosure. FIG. 1C provides a more detailed view of the cohort generation module 126 and its relationship with the cohort generation algorithm 127 within the cohort generation platform 120. As depicted, the cohort generation module 126 includes a cohort generation algorithm 127. The cohort generation algorithm 127 functions to determine appropriate populations, such as eligible populations, representative categories, sample cohorts, cohort rankings, or the like, to provide to a user based on various factors, such as population data 110. Furthermore, the cohort generation algorithm 127 also takes into account the performance of past generated populations in similar contexts to increase the likelihood of a favorable user response.

The cohort generation algorithm 127, as part of the cohort generation module 126, orchestrates the creation of cohorts from the population data 110. This algorithm is agnostic to its underlying implementations and is designed to accommodate various types of algorithms, either individually or in combination, to achieve the desired outcomes. In some embodiments, the cohort generation algorithm 127 operates as a divergence algorithm. In this context, the divergence algorithm calculates a deviation measure, such as a measure of the statistical divergence between different populations, subgroups, and sample cohorts within the population data 110. The algorithm performs this by evaluating the representation of various features or attributes within each group and comparing it with the representation of the same features or attributes in the overall population or other subgroups. The deviation measure, often expressed as a divergence value, assists in generating the true eligible population and the representative categories, as it provides a quantitative assessment of how well a given sample represents the overall population.

In some embodiments, the cohort generation algorithm 127 represents a collection of distinct algorithms, each invoked at discrete steps within the cohort generation process. For instance, one step may employ a clustering algorithm to group individuals in the population data 110 based on shared attributes. Another step may utilize a classification algorithm to assign individuals to the true eligible population based on the eligibility criteria. Yet another step may leverage a ranking algorithm to prioritize the sample populations based on their divergence values. Further, one or more of these discrete steps within the cohort generation algorithm 127, in some embodiments, employs the use of a machine learning model. Machine learning models, such as decision trees, neural networks, or support vector machines, enable the algorithm to learn from the population data 110 and improve its ability to generate true eligible populations and representative categories over time. For example, a supervised learning model, in some embodiments, is trained on population data, to learn the associations between individual attributes and cohort eligibility, thereby increasing the accuracy and efficiency of the cohort generation process.

The cohort generation algorithm 127 is designed to be flexible and adaptable, capable of incorporating different algorithmic approaches or machine learning models depending on the specific requirements of the cohort generation task. It is this adaptability that allows the cohort generation algorithm 127 to effectively handle the complexities present in dealing with diverse and dynamic population data 110.

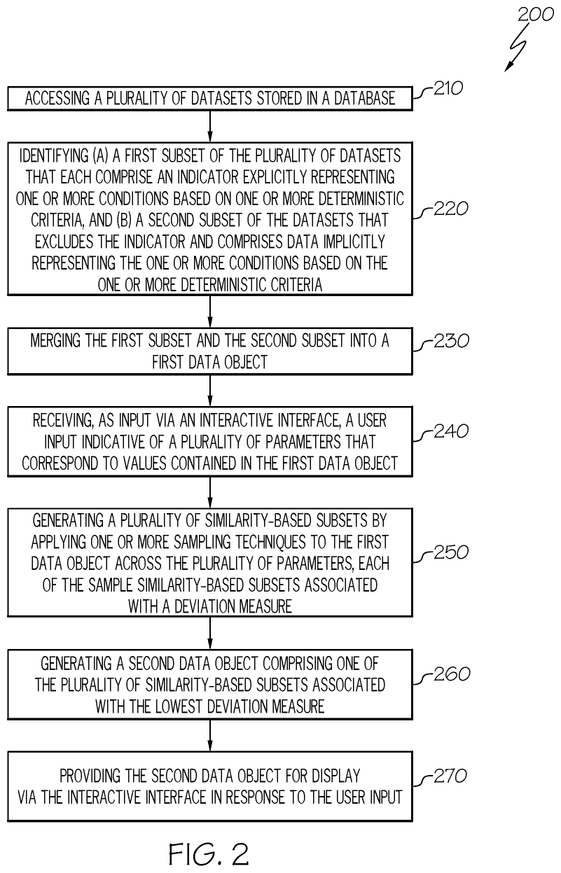

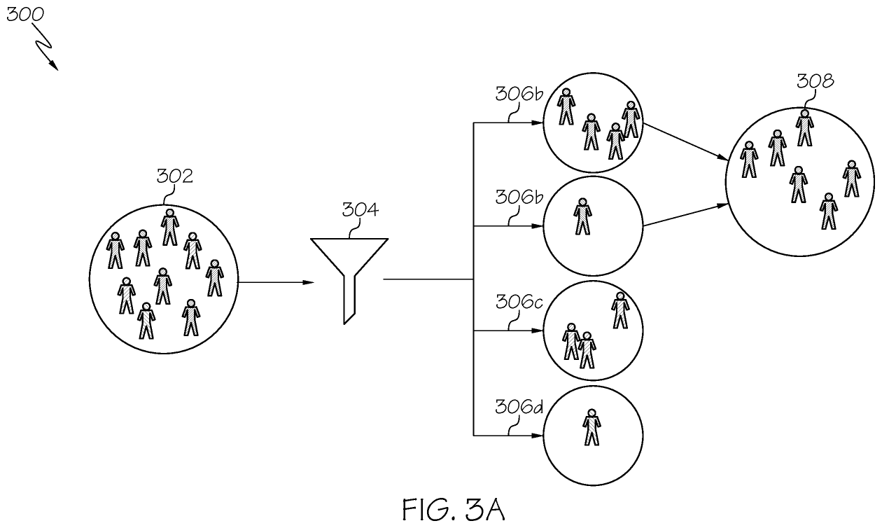

FIG. 2 is a flowchart showing a method cohort generation, according to some embodiments of the disclosure. In one embodiment, method 200 may be performed by the cohort generation platform 120. Step 210 involves accessing a plurality of datasets stored in a database. The cohort generation platform 120 may receive one or more dataset, such as by receiving a first population (e.g., a first group), wherein the first population is associated with one or more healthcare records. The dataset can comprise a collection of individuals associated with healthcare data contained in electronic medical records, insurance claims data, or other types of healthcare records stored in one or more databases, such as population data 110.

In some embodiments, the first population of the dataset includes a plurality of members, each member representing a unique individual associated with healthcare records. The healthcare records may contain structured and/or unstructured data related to demographics, medical history, diagnoses, procedures, medications, immunizations, allergies, radiology images, laboratory test results, genomics data, vital signs, insurance claims, socioeconomic factors, and other healthcare information for each individual.

The cohort generation platform 120, in some embodiments, retrieves the plurality of datasets, such as population data 110, from various sources including hospitals, clinics, pharmacies, laboratories, health information exchanges, payers, government agencies, research institutions, and other healthcare-related entities. The dataset can be stored in relational databases, non-relational databases, data warehouses, cloud-based storage, file systems, or other computer-based storage mechanisms, each of which may be referred to as database 125. Data exchange interfaces like HL7 FHIR may be used to access the first population health data.

In some embodiments, the cohort generation platform 120 is configured to accept populations of varied size and varied numbers of associated healthcare records. The dataset, which includes first population data, provides the raw data that will be analyzed and filtered in subsequent steps to generate one or more representative sample cohorts.

Step 220 involves identifying (a) a first subset of the plurality of datasets that each comprise an indicator explicitly representing one or more conditions based on one or more deterministic criteria, and (b) a second subset of the datasets that excludes the indicators and comprises data implicitly representing the one or more conditions based on the one or more deterministic criteria. In some embodiments, the identification includes defining a plurality of eligibility criteria, such as the presence of one or more indicators, which in some embodiments are based on one or more categories associated with a medical study. For example, an eligibility criteria may require that data associated with a member include one or more indicators, and in some embodiments the indicators may include data indicating that the member has been diagnosed with one or more conditions. Each eligibility criteria serves as a rule to define one or more subgroups associated with the first population. Each subgroup contains members which are unique when compared against members of another subgroup.

For example, in some embodiments, a first subset of the dataset includes one or more indicators, and/or in some embodiments data associated with the members of the subset include one or more indicators, that the subset includes individuals (members) which have been diagnosed with a specific condition relevant to the study, as identified by diagnosis codes in their healthcare records. By way of another example, a second subgroup includes data, and/or includes data associated with the members of the subset, which implies individuals (members) which have been determined, without a formal diagnosis, to likely have the condition based on other clinical data in their records, with the condition being assigned to each individual through deterministic criteria. Additional subgroups are defined using similar eligibility criteria and/or indicators tailored to the requirements of the study.

Step 230 includes merging the first subset and the second subset into a first data object. At this step, once all desired subgroups and/or subsets have been identified in the plurality of datasets (such as the population data 110), one or more subsets (such as the first subset and the second subset) are combined to generate the first data object, which may be representative of a true eligible population target that meets all the eligibility criteria. Cohort generation platform 120 identifies one or more subgroups and/or subsets where the defining eligibility criteria of the one or more subgroups indicate that the subgroup is eligible for the trial, such as the first subset (comprising the indicator) and the second subset (comprising data which implies a specific condition). Once all eligible subgroups are identified, the eligible subgroups are combined into a first data set, which is a true eligible population. This true eligible population represents the total pool matching the study requirements before sampling.

After the true eligible population is generated in Step 220, this population can optionally be further filtered in an additional step to identify a willing population, which then may be merged into the first data object. The willing population comprises members of the true eligible population who have indicated willingness to participate in clinical studies, based on factors such as opt-in status, past participation, declared interest, and lack of opt-out status.

In some embodiments, the remaining steps utilize this first data object (either as the true eligible population or the willing population) as the starting point for generating a plurality of similarity-based subsets, such as sample cohorts, allowing the similarity-based subsets to better reflect engaged participants. In other embodiments, the willing population filter is not applied, and the true eligible population is used directly as the starting point in subsequent steps. In such embodiments, one or more additional data objects are generated for each population. This allows the subsets to maintain representation of the entire eligible population, regardless of expressed willingness. Therefore, the true eligible population produced in Step 220, in both filtered and unfiltered configurations, may be deemed the first data object utilized in the systems and methods described herein. The approach taken is adaptable to the requirements of the particular study.

Step 240 involves receiving, as input via an interactive interface, a user input indicative of a plurality of parameters that correspond to values contained in the first data object. The user input may be received through user interface module 128, and may be indicative of one or more parameters or attributes applicable to the population that, in some embodiments, exhibit variation across the individual members of each data object, such as a true willing population. For examples, the parameters include demographics like age, gender, ethnicity, income level; geographic region; healthcare utilization metrics; costs; sites of care; providers; chronic conditions; prescription drug use; lab results; or the like.

In some embodiments, the system and method include generating a plurality of representative categories for the true eligible population or willing population. This generation, in some embodiments, is performed by the cohort generation platform by utilizing the plurality of parameters, identifying one or more options associated with each of the plurality of parameters, and identifying one or more, or all, combinations of options associated with each parameter to define a plurality of representative categories. Each unique combination of parameter options is, in some embodiments, a unique representative category.

For each selected parameter, the process identifies a set of mutually exclusive options or bins encompassing all possible values for that parameter. For example, for an ‘Age’ parameter, the options could be 18-30, 31-40, 41-50, 51-60, 61-70, 71-80, and 81+. For a ‘Region’ parameter, the options could be Northeast, Southeast, Midwest, Southwest, and West. With the options defined for each parameter, the process then systematically generates all possible combinations of options across the parameters. Each unique combination represents one representative category that members of the population could fall into. With just 3 binary parameters, there would be 2×2×2=8 categories. With 5 parameter each having 5 options, there would be 5×5×5×5×5=3,125 categories. The number of categories expands exponentially as more parameter and options are added to provide finer-grained resolution.

The set of representative categories represents the full cross-product covering the variation present in the first data object, which represents the true eligible or willing population. Each member of the population falls into one and only one category based on their specific values for the defined parameters. This categorical representation of the population is leveraged in subsequent steps, and may be referred to as ‘binning’ or producing a plurality of bins.

The cohort generation platform, in some embodiments, determines which parameters to select for generating representative categories through approaches including but not limited to statistical analysis of the population data to identify attributes exhibiting significant variance, leveraging domain expertise from medical researchers to select clinically relevant covariates, receiving data associated with published literature to identify impactful criteria suggested by prior research, employing automated feature selection techniques like principal component analysis to analyze the data and discover relationships, visually and statistically exploring the data to reveal underlying patterns that inform parameters selection, and training proxy predictive models on the population using candidate parameters and selecting those with the highest feature importance—with the goal of identifying optimal parameters able to meaningfully stratify the population according to the specifics of the data and aims of the medical study.

Step 250 involves generating a plurality of similarity-based subsets by applying one or more sampling techniques to the first data object across the plurality of parameters, each of the sample similarity-based subsets associated with a deviation measure. The generation of the plurality of similarity-based subsets includes, in some embodiments, selecting a plurality of sample populations (e.g., a plurality of sample groups, or similarity-based subsets), each of the plurality of sample populations being a subset of the first data object, which in some embodiments is the true eligible population. Various probabilistic sampling techniques can be utilized to draw representative and/or similarity-based subsets without bias. In one embodiment, simple random sampling assigns members random numbers, shuffles them, and takes the first N as a sample based on desired size N. This gives equal selection probability. In another embodiment, stratified random sampling groups the population of the first data object into strata by key criteria, sampling proportionally from each strata to maintain representation across important subgroups.

Additional techniques like cluster, systematic, reservoir, and weighted sampling may be employed per computational efficiency, statistical properties, and other factors. The sampling method is chosen to best fit the data and goals. In some embodiments, Monte Carlo sampling is used to randomly select sample members in a way that allows robust statistical analysis of the samples' representative accuracy.

In some embodiments, multiple sample populations (which in some embodiments are similarity-based subsets) are selected, each with equal number of members. For instance, 10 subsets containing 10,000 members each may be generated. Multiple subsets allow assessment of variability to determine one or more optimal subsets. The number of subsets and size of each subsets can be tuned as parameters, set sufficiently large to represent population variation while remaining computationally practical. Appropriate probabilistic sampling and adequate sample sizes help ensure the resulting subsets and/or cohorts accurately represent the true eligible or willing population contained within the first data object for recruitment purposes.

In some embodiments, each similarity-based subset is associated with a divergence metric. As will be discussed, the divergence metric is, in some embodiments, a representation of the amount the similarity-based subset deviates and/or diverges from the first data object, such as the true eligible population. In some embodiments, the divergence metric is the result of one or more divergence calculations.

In one embodiment, the results of the divergence calculations performed on earlier similarity-based subsets is leveraged to inform and optimize selection of subsequent similarity-based subsets. For example, the representation criteria and strata that exhibited high divergence in prior similarity-based subsets could be further emphasized in later sampling by adjusting their selection probabilities. The categories identified as having greater divergence from the first data object distribution in previous iterations can be specifically targeted for improved representation in newer similarity-based subsets.

By analyzing earlier divergence results, the sampling can become iterative and self-correcting, progressively improving the selection of representative similarity-based subset over time. This creates a feedback loop that concentrates sampling power where it is most needed to minimize divergence. Such informed sampling techniques can increase efficiency by reducing redundant computations, allowing the process to converge on optimal similarity-based subset faster. The computational burden of excessive random sampling can be avoided by intelligently focusing the sampling where it will be most impactful.

In one embodiment, the results of the divergence calculations performed on earlier similarity-based subsets are utilized to inform the selection of subsequent similarity-based subsets using Monte Carlo sampling methodology, wherein each member of the first data object (the true eligible population) is assigned a random selection probability that is dynamically weighted based on the divergence results of said member's representative category in previous similarity-based subsets, such that members of underrepresented categories with high divergence have increased selection probabilities boosted to make them more likely to be randomly selected for inclusion to improve representation, while members of overrepresented categories with low divergence have decreased selection probabilities accordingly, and whereby performing iterative rounds of weighted Monte Carlo sampling allows the process to leverage the feedback from prior divergence calculations to correct for deficiencies by stochastically converging on more representative samples through continuously adjusted probabilistic sampling focused on capturing those categories difficult to sample.

In some embodiments, the system and method further include determining a similarity-based subset divergence value, which may be a deviation measure for each of the plurality of similarity-based subsets. This includes, for each representative category of each respective sample population, determining a probability of that category being selected from the true eligible population contained in the first data object, determining a probability of that category and/or parameter being selected from the respective similarity-based subsets, and generating a divergence value for the category based on comparing the probability of selection from the true population versus the similarity-based subsets.

The probability of a category being selected from the first data object is calculated by dividing the number of members from the true eligible population that fall into that category by the total size of the true eligible population. Likewise, the probability of category selection from the similarity-based subset is based on the portion of the similarity-based subset in that category.

A deviation measure is generated for each category using a mathematical measure of divergence like Kullback-Leibler divergence, Euclidean distance, or other distance statistic that quantifies the difference between two probability distributions. For example, if a category has a 10% selection probability in the true population but only 5% selection probability in the sample, the divergence value for that category would indicate the under-representation. The greater the divergence between the true and sample category probabilities, the higher the divergence value.

Once a divergence value has been calculated for every representative category, these values are aggregated to generate an overall similarity-based subset divergence value. The aggregation can be done by summing, averaging, or taking a maximum of the individual category divergences. It results in a single composite divergence score that reflects how well the similarity-based subset matches the true population distribution across all categories.

By repeating this process for each similarity-based subset, a deviation measure is obtained for each one. The deviation measures allow quantitative comparison of the representativeness of the different similarity-based subsets to inform selection of the best similarity-based subsets. In some embodiments, lower divergence indicates higher accuracy in reproducing the true population distribution.

In some embodiments, while calculating the deviation measure, the cohort generation platform assigns a weight to each representative category, or ‘bin’, that reflects its significance or relevance in the context of the study. The weight is related to the category's importance, its variance within the population, or other domain-specific considerations. For example, a category with a higher frequency in the true population may be given more weight, as its misrepresentation could have a larger impact on the results. Alternatively, categories that are of particular interest or significance in the context of the research question may be weighted more heavily. When calculating the divergence value for a category, the probability differences are multiplied by the weight of the category. Alternatively, in the case that a user desires to intentionally exclude or lessen certain categories, lower or negative weights are applied. By incorporating these weights into the divergence calculation, the overall similarity-based subset divergence value reflects not only the raw differences between similarity-based subset and true population distributions, but also the relative importance of these differences across the various categories. This provides a more nuanced and context-sensitive measure of the representativeness of each similarity-based subset.

Step 260 involves generating a second data object comprising one of the plurality of similarity-based subsets associated with the lowest deviation measure. The second data object, in some embodiments, contains only the similarity-based subset that has the lowest deviation measure, while in some embodiments, the second data object includes a plurality of the plurality of the similarity-based subsets, up to the second data object including every similarity-based subset and their associated deviation measure. In some embodiments, the method includes ranking the plurality of similarity-based subsets based on the deviation measure determined for each of the plurality of similarity-based subsets. This enables determining the optimal similarity-based subset that most closely matches the true eligible population or willing population distribution associated with the first data object. With the deviation measure computed, the similarity-based subsets are ranked from lowest to highest divergence. The similarity-based subset with the lowest divergence value is considered the most representative of the true population, as its category probability distribution most closely aligns with the true distribution. Higher divergence indicates more deviation from the true probabilities.

In one embodiment, the ranking is performed within defined member size categories, such that samples of similar sizes are compared directly. The member size categories each represent a sample size range (e.g. 5,000-10,000 members, 10,000-20,000 members, etc.), or may represent specific sizes (e.g., 10 members, 11, members, 12 members, and so on). Within each size category, the similarity-based subsets are ranked by divergence, identifying the similarity-based subset with lowest divergence score. This allows determining an optimal similarity-based subset for each desired size tier. The size-based ranking accounts for differences in divergence across sizes. Smaller similarity-based subsets may exhibit more variance simply due to their size. The divergence value of the top ranked sample within each size category can be compared to a defined threshold divergence value to determine whether similarity-based subsets of that size are satisfactory. If the divergence is below the threshold, that size category is considered acceptable.

The ranking and threshold comparison identifies the optimal similarity-based subset(s) across the desired range of sizes that match the true population with divergence scores meeting the required representativeness criteria. These samples represent the highest quality cohorts for the intended study recruiting. Advantageously, this allows users, such as researches, to define an acceptable threshold of divergence and then identify the cohort with the smallest sample size which meets the acceptable threshold of divergence.

Step 270 involves providing the second data object for display via the interactive interface in response to the user input. In some embodiments, the second data object is provided along with information about the deviation measures associated with it and/or other similarity-based subsets. In some embodiments, the user can view the deviation measures and select the most appropriate similarity-based subset for their needs. Additionally, the user can compare the deviation measures of multiple similarity-based subsets side-by-side to make an informed decision about which one to choose. Furthermore, the user can adjust the threshold values for the deviation measures based on their preferences, allowing them to customize the selection process according to their requirements. In some embodiments, the interactive interface displays a visual indicator of member size categories that satisfy a threshold value, enabling users to quickly identify groups of members that meet their criteria.

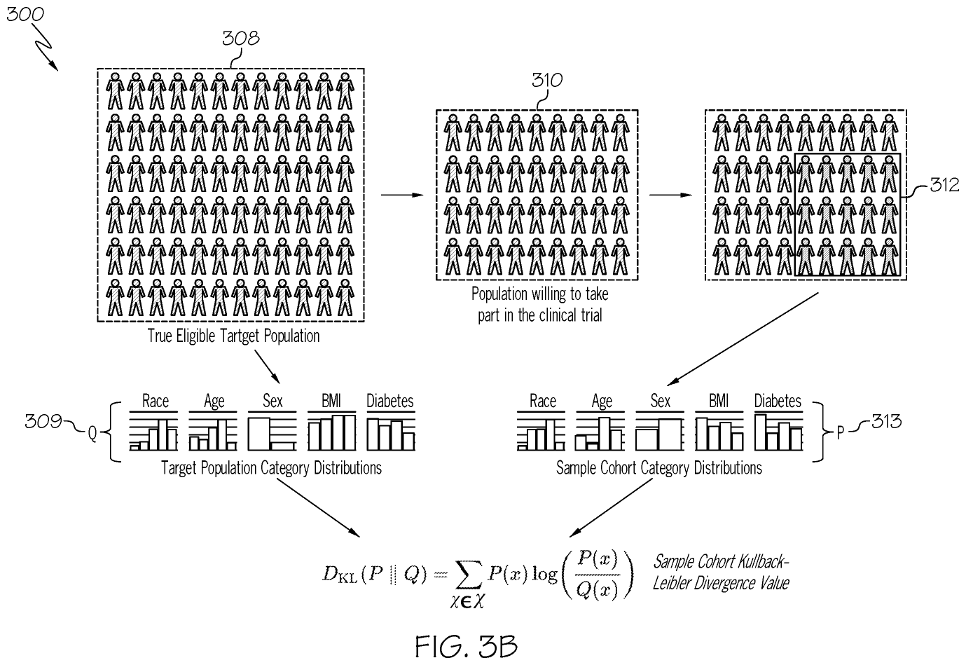

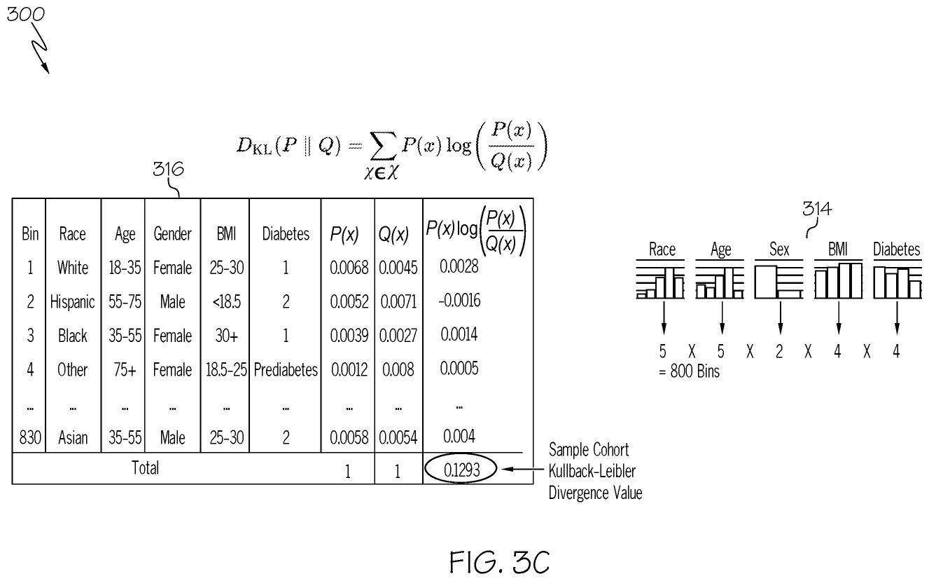

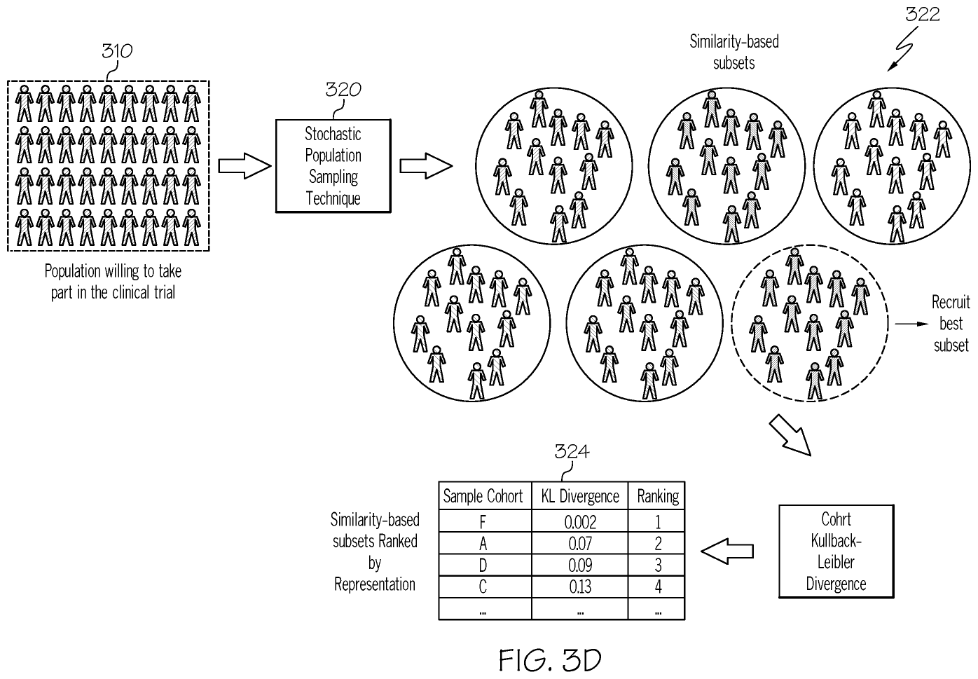

FIG. 3A is a flowchart showing one or more steps in a method for generating a similarity-based subset, according to some embodiments of the disclosure. FIG. 3A provides an overview of the initial stages in the example process 300 for similarity-based subset generation. Specifically, it illustrates the stages involving the entire population, application of one or more filters for stratification, generation of resulting subgroups, and the identification of a true eligible target population, which may be then merged as a first data object.

The process begins with the population 302, which represents the comprehensive group of individuals from which a cohort for a medical study or investigation is to be selected. This entire population 302 may encompass a broad array of demographic, clinical, genetic, and other data as discussed herein, and it is typically stored within one or more dataset, such as the population data 110, as represented in FIG. 1A.

Following the identification of the entire population 302, the process applies one or more filters 304 for stratification. These filters 304 are criteria or guidelines used to segregate the population 302 into distinct subgroups. These filters may be based on a variety of characteristics, such as age, gender, medical history, genetic markers, lifestyle factors, or the like. The application of these filters 304 is handled by the cohort generation platform 120, and in some embodiments particularly the data collection module 122 and data processing module 124.

Once the filters 304 have been applied, the process generates one or more resulting subsets of the dataset, such as subgroups 306a, 306b, 306c, and 306d. Each subgroup represents a distinct segment of the entire population 302 that shares common characteristics as defined by the applied filters. These subgroups are stored within the database 125 of the cohort generation platform 120.

The final stage depicted in FIG. 3A involves the identification of a true eligible target population 308 for inclusion in the first data object. The true eligible target population 308 is a subset of the population 302 that meets specific eligibility criteria for inclusion in the medical study or investigation. These criteria may relate to factors such as the presence of a specific medical condition, the absence of certain exclusionary factors, or the like. The identification of the true eligible target population 308 is performed by the cohort generation platform 120, specifically via the cohort generation module 126. As shown in FIG. 3A, in some embodiments not all subgroups are deemed to be part of the true eligible target population 308, such that one or more subgroup is excluded.

The stages and components described above in the context of FIG. 3A can be arranged in various sequences and combinations to achieve the overall goal of generating a true eligible target population from a larger entire population. The exact implementation can vary depending on the specific requirements and constraints of the study or investigation being conducted.

FIG. 3B is a flowchart showing one or more steps in a method for generating a similarity-based subset, according to some embodiments of the disclosure. FIG. 3B demonstrates a segment of the exemplary process 300 for similarity-based subset generation, specifically focusing on the true eligible target population and the generation of a willing population and sample similarity-based subset populations, along with their respective category distributions. The figure begins with the true eligible target population 308. As previously described, the true eligible target population 308 is a subset of the entire population that satisfies the specific eligibility criteria for participation in a given medical study, and may be associated with or within a first data object. The true eligible target population 308 is characterized by its category distribution 309, which represents the distribution of members across various categories or bins as defined by certain demographic, clinical, or genetic factors, or the like. The categories, in some embodiments, are related to the one or more parameters as previously discussed.

Once the true eligible target population 308 and its category distribution 309 have been established, the process proceeds to identify a willing population 310. The willing population 310 is a subset of the true eligible target population 308 that has indicated a willingness or availability to participate in the medical study. This willingness may be determined through direct contact and consent, through the application of additional selection criteria, or through other appropriate means. The identification of the willing population 310 is managed by the cohort generation platform 120, specifically through the use of the data collection module 122 and/or user interface module 128.

Following the identification of the willing population 310, a similarity-based subset 312 is selected. The similarity-based subset 312 is a subset of the willing population 310 that is selected as a sample participation group in the medical study. The selection of the similarity-based subset 312 is executed by the cohort generation platform 120, specifically by the cohort generation module 126, and is typically based on a sampling technique that aims to ensure the similarity-based subset 312 is representative of the true eligible target population 308, as described herein.

Finally, the similarity-based subset 312 is characterized by its category distribution 313. The category distribution 313 represents the distribution of members across various categories or bins within the similarity-based subset 312. The category distribution 313 is compared with the category distribution 309 of the true eligible target population 308 to assess the representativeness of the similarity-based subset 312. This comparison is executed by the cohort generation platform 120, specifically by the data processing module 124.

As with the stages and components described in connection with FIG. 3A, the stages and components in FIG. 3B can be executed in various sequences and combinations, depending on the specific requirements and constraints of the medical study or investigation.

FIG. 3C is a flowchart showing one or more steps in a method for generating a similarity-based subset, according to some embodiments of the disclosure. FIG. 3C illustrates a particular portion of an example process 300 for similarity-based subset generation, specifically focusing on bin determination, bin evaluation, and sample divergence value calculation. The first stage in this figure is the bin determination 314. Bin determination 314, in the context of the present invention, refers to the process of classifying the members of a first data object, such as a true eligible target population, into various representative categories or “bins”. The bins can be defined based on various demographic, clinical, and/or genetic factors, or the like. Each bin represents a distinct group within the population that shares one or more common characteristics defined by the binning criterion. The bin determination 314 can be executed by the cohort generation platform 120 utilizing the cohort generation module 126, or more specifically, the cohort generation algorithm 127.

Following bin determination 314, the process moves to bin evaluation 316. Bin evaluation 316 involves assessing the composition of each bin resulting from the bin determination 314. In some embodiments, this includes counting the number of members in each bin, calculating the proportion of the total population that each bin represents, assessing the distribution of various characteristics within each bin, or the like. The bin evaluation 316 is performed to understand the structural composition of the target population and to provide an empirical basis for the subsequent generation of sample similarity-based subsets. This evaluation can also be performed by the cohort generation platform 120, more specifically by the data processing module 124.

The final step in this figure is the calculation of the sample divergence value 318. The sample divergence value 318 serves as a quantitative measure of how a given similarity-based subset diverges from the true eligible target population in terms of the distribution of members across the various bins. In some embodiments, this is performed by the cohort generation platform using various statistical measures such as the Kullback-Leibler divergence, total variation distance, Jensen-Shannon divergence, or the like. The sample divergence value 318 is a metric that enables the ranking of different similarity-based subsets based on their representativeness of the true eligible target population. This calculation is also executed by the cohort generation platform 120, specifically using the data processing module 124.

As with the stages and components described in connection with FIG. 3A and FIG. 3B, the components and stages described above in the context of FIG. 3C can be arranged in various combinations and sequences while still achieving the same overall objective of generating representative sample cohorts from a target population. The precise implementation may vary depending on the specific requirements and constraints of the cohort generation task at hand.

FIG. 3D is a flowchart showing one or more steps in a method for generating a similarity-based subset, according to some embodiments of the disclosure. FIG. 3D illustrates a subsequent segment of the example process 300 for similarity-based subset generation, specifically the stages involving the willing population, the application of a sampling technique, the generation of sample cohorts, and the ranking of these sample similarity-based subsets.

The first stage in this figure, the willing population 310, represents a subset of the true eligible target population that is amenable to participation in the medical study or investigation. The willing population 310 is typically identified through a process of obtaining consent from the members of the true eligible target population, or alternatively, it may result from the application of certain selection criteria, such as the availability or accessibility of members, their willingness to participate, or the like. This stage is executed by the cohort generation platform 120, specifically using the data collection module 122.

Following the identification of the willing population 310, the process moves to the application of a sampling technique 320. The sampling technique 320 refers to a method or algorithm for selecting a subset of members from the willing population 310 to form a similarity-based subset. Various sampling techniques, as discussed, may be employed, such as simple random sampling, stratified sampling, systematic sampling, cluster sampling, Monte Carlo sampling, or the like. The choice of sampling technique depends on factors such as the size and diversity of the willing population 310, the desired size and composition of the similarity-based subset, the nature of the medical study, or other considerations. The sampling technique 320 is executed by the cohort generation platform 120, specifically by the cohort generation module 126.

The application of the sampling technique 320 results in the generation of one or more sample similarity-based subsets 322. Each sample similarity-based subset represents a distinct group of members selected from the willing population 310. Each sample similarity-based subset 322 is intended to be representative of the willing population and, by extension, the true eligible target population. The generation of the sample similarity-based subset 322 is managed by the cohort generation platform 120, specifically by the cohort generation module 126.

The final step in FIG. 3D is the ranking of the sample similarity-based subset 324. This ranking is based on the sample divergence values calculated in the preceding stage (as depicted in FIG. 3C). The sample similarity-based subsets 322 are ranked in ascending order of their sample divergence values, such that the sample similarity-based subset with the lowest divergence value (i.e., the most representative of the true eligible target population) is ranked highest. The ranking of the sample similarity-based subset 324 is performed by the cohort generation platform 120, specifically by the data processing module 124.

As with the components and stages described in connection with FIGS. 3A, 3B, and 3C, the components and stages described above in the context of FIG. 3D can be arranged in various sequences and combined in different ways, depending on the specific requirements and constraints of the cohort generation task.

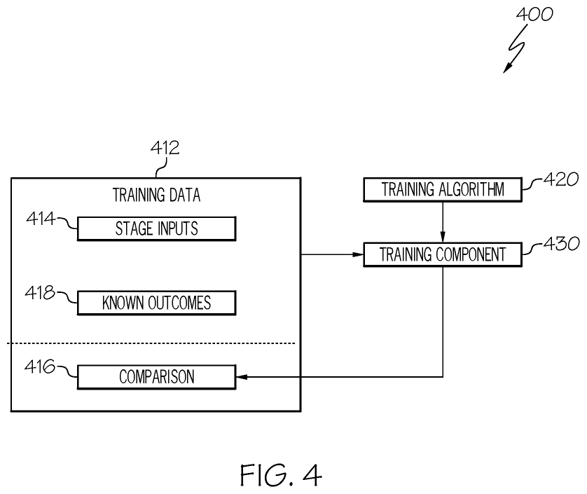

One or more implementations disclosed herein include and/or are implemented using a machine-learning model. For example, one or more of the modules of the cohort generation platform are implemented using a machine-learning model and/or are used to train the machine-learning model. FIG. 4 shows an example machine-learning training flow chart, according to some embodiments of the disclosure. Referring to FIG. 4, a given machine-learning model is trained using the training flow chart 400. The training data 412 includes one or more of stage inputs 414 and the known outcomes 418 related to the machine-learning model to be trained. The stage inputs 414 are from any applicable source including text, visual representations, data, values, comparisons, and stage outputs, e.g., one or more outputs from one or more steps from FIG. 2-FIG. 3D. The known outcomes 418 are included for the machine-learning models generated based on supervised or semi-supervised training, or can based on known labels, such as topic labels. An unsupervised machine-learning model is not trained using the known outcomes 418. The known outcomes 418 includes known or desired outputs for future inputs similar to or in the same category as the stage inputs 414 that do not have corresponding known outputs.

The training data 412 and a training algorithm 420, e.g., one or more of the modules implemented using the machine-learning model and/or are used to train the machine-learning model, is provided to a training component 430 that applies the training data 412 to the training algorithm 420 to generate the machine-learning model. According to an implementation, the training component 430 is provided comparison results 416 that compare a previous output of the corresponding machine-learning model to apply the previous result to re-train the machine-learning model. The comparison results 416 are used by the training component 430 to update the corresponding machine-learning model. The training algorithm 420 utilizes machine-learning networks and/or models including, but not limited to a deep learning network such as Deep Neural Networks (DNN), Convolutional Neural Networks (CNN), Fully Convolutional Networks (FCN) and Recurrent Neural Networks (RCN), probabilistic models such as Bayesian Networks and Graphical Models, classifiers such as K-Nearest Neighbors, and/or discriminative models such as Decision Forests and maximum margin methods, the model specifically discussed herein, or the like.

The machine-learning model used herein is trained and/or used by adjusting one or more weights and/or one or more layers of the machine-learning model. For example, during training, a given weight is adjusted (e.g., increased, decreased, removed) based on training data or input data. Similarly, a layer is updated, added, or removed based on training data/and or input data. The resulting outputs are adjusted based on the adjusted weights and/or layers.

In general, any process or operation discussed in this disclosure is understood to be computer-implementable, such as the process illustrated in FIGS. 2-3D are performed by one or more processors of a computer system as described herein. A process or process step performed by one or more processors is also referred to as an operation. The one or more processors are configured to perform such processes by having access to instructions (e.g., software or computer-readable code) that, when executed by one or more processors, cause one or more processors to perform the processes. The instructions are stored in a memory of the computer system. A processor is a central processing unit (CPU), a graphics processing unit (GPU), or any suitable type of processing unit.

A computer system, such as a system or device implementing a process or operation in the examples above, includes one or more computing devices. One or more processors of a computer system are included in a single computing device or distributed among a plurality of computing devices. One or more processors of a computer system are connected to a data storage device. A memory of the computer system includes the respective memory of each computing device of the plurality of computing devices.

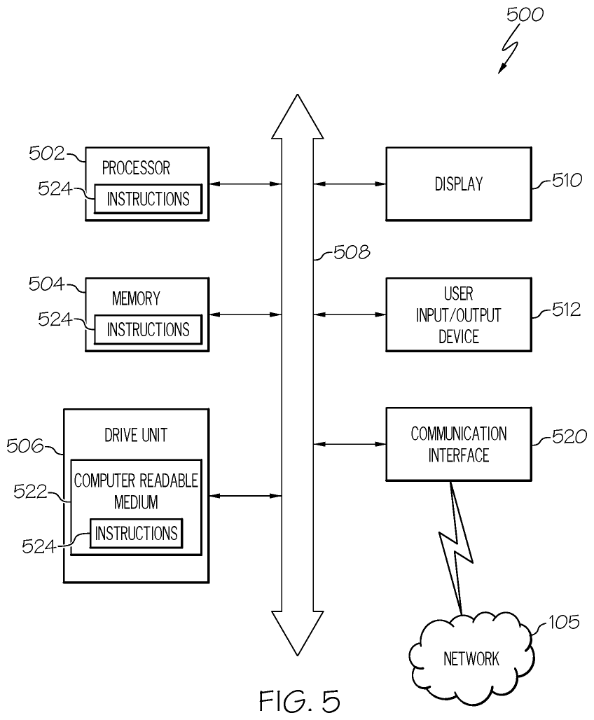

FIG. 5 illustrates an implementation of a computer system that executes techniques presented herein. The computer system 500 includes a set of instructions that are executed to cause the computer system 500 to perform any one or more of the methods or computer based functions disclosed herein. The computer system 500 operates as a standalone device or is connected, e.g., using a network, to other computer systems or peripheral devices.

Unless specifically stated otherwise, as apparent from the following discussions, it is appreciated that throughout the specification, discussions utilizing terms such as “processing,” “computing,” “calculating,” “determining”, analyzing” or the like, refer to the action and/or processes of a computer or computing system, or similar electronic computing device, that manipulate and/or transform data represented as physical, such as electronic, quantities into other data similarly represented as physical quantities.

In a similar manner, the term “processor” refers to any device or portion of a device that processes electronic data, e.g., from registers and/or memory to transform that electronic data into other electronic data that, e.g., is stored in registers and/or memory. A “computer,” a “computing machine,” a “computing platform,” a “computing device,” or a “server” includes one or more processors.

In a networked deployment, the computer system 500 operates in the capacity of a server or as a client user computer in a server-client user network environment, or as a peer computer system in a peer-to-peer (or distributed) network environment. The computer system 500 is also implemented as or incorporated into various devices, such as a personal computer (PC), a tablet PC, a set-top box (STB), a personal digital assistant (PDA), a mobile device, a palmtop computer, a laptop computer, a desktop computer, a communications device, a wireless telephone, a land-line telephone, a control system, a camera, a scanner, a facsimile machine, a printer, a pager, a personal trusted device, a web appliance, a network router, switch or bridge, or any other machine capable of executing a set of instructions (sequential or otherwise) that specify actions to be taken by that machine. In a particular implementation, the computer system 500 is implemented using electronic devices that provide voice, video, or data communication. Further, while the computer system 500 is illustrated as a single system, the term “system” shall also be taken to include any collection of systems or sub-systems that individually or jointly execute a set, or multiple sets, of instructions to perform one or more computer functions.

As illustrated in FIG. 5, the computer system 500 includes a processor 502, e.g., a central processing unit (CPU), a graphics processing unit (GPU), or both. The processor 502 is a component in a variety of systems. For example, the processor 502 is part of a standard personal computer or a workstation. The processor 502 is one or more processors, digital signal processors, application specific integrated circuits, field programmable gate arrays, servers, networks, digital circuits, analog circuits, combinations thereof, or other now known or later developed devices for analyzing and processing data. The processor 502 implements a software program, such as code generated manually (i.e., programmed).

The computer system 500 includes a memory 504 that communicates via bus 508. The memory 504 is a main memory, a static memory, or a dynamic memory. The memory 504 includes, but is not limited to computer-readable storage media such as various types of volatile and non-volatile storage media, including but not limited to random access memory, read-only memory, programmable read-only memory, electrically programmable read-only memory, electrically erasable read-only memory, flash memory, magnetic tape or disk, optical media and the like. In one implementation, the memory 504 includes a cache or random-access memory for the processor 502. In alternative implementations, the memory 504 is separate from the processor 502, such as a cache memory of a processor, the system memory, or other memory. The memory 504 is an external storage device or database for storing data. Examples include a hard drive, compact disc (“CD”), digital video disc (“DVD”), memory card, memory stick, floppy disc, universal serial bus (“USB”) memory device, or any other device operative to store data. The memory 504 is operable to store instructions executable by the processor 502. The functions, acts, or tasks illustrated in the figures or described herein are performed by the processor 502 executing the instructions stored in the memory 504. The functions, acts, or tasks are independent of the particular type of instruction set, storage media, processor, or processing strategy and are performed by software, hardware, integrated circuits, firmware, micro-code, and the like, operating alone or in combination. Likewise, processing strategies include multiprocessing, multitasking, parallel processing, and the like.

As shown, the computer system 500 further includes a display 510, such as a liquid crystal display (LCD), an organic light emitting diode (OLED), a flat panel display, a solid-state display, a cathode ray tube (CRT), a projector, a printer or other now known or later developed display device for outputting determined information. The display 510 acts as an interface for the user to see the functioning of the processor 502, or specifically as an interface with the software stored in the memory 504 or in the drive unit 506.

Additionally or alternatively, the computer system 500 includes an input/output device 512 configured to allow a user to interact with any of the components of the computer system 500. The input/output device 512 is a number pad, a keyboard, a cursor control device, such as a mouse, a joystick, touch screen display, remote control, or any other device operative to interact with the computer system 500.

The computer system 500 also includes the drive unit 506 implemented as a disk or optical drive. The drive unit 506 includes a computer-readable medium 522 in which one or more sets of instructions 524, e.g. software, is embedded. Further, the sets of instructions 524 embodies one or more of the methods or logic as described herein. The sets of instructions 524 resides completely or partially within the memory 504 and/or within the processor 502 during execution by the computer system 500. The memory 504 and the processor 502 also include computer-readable media as discussed above.

In some systems, computer-readable medium 522 includes the set of instructions 524 or receives and executes the set of instructions 524 responsive to a propagated signal so that a device connected to network 105 communicates voice, video, audio, images, or any other data over the network 105. Further, the sets of instructions 524 are transmitted or received over the network 105 via the communication port or interface 520, and/or using the bus 508. The communication port or interface 520 is a part of the processor 502 or is a separate component. The communication port or interface 520 is created in software or is a physical connection in hardware. The communication port or interface 520 is configured to connect with the network 105, external media, the display 510, or any other components in the computer system 500, or combinations thereof. The connection with the network 105 is a physical connection, such as a wired Ethernet connection, or is established wirelessly as discussed below. Likewise, the additional connections with other components of the computer system 500 are physical connections or are established wirelessly. The network 105 alternatively be directly connected to the bus 508.

While the computer-readable medium 522 is shown to be a single medium, the term “computer-readable medium” includes a single medium or multiple media, such as a centralized or distributed database, and/or associated caches and servers that store one or more sets of instructions. The term “computer-readable medium” also includes any medium that is capable of storing, encoding, or carrying a set of instructions for execution by a processor or that causes a computer system to perform any one or more of the methods or operations disclosed herein. The computer-readable medium 522 is non-transitory, and may be tangible.

The computer-readable medium 522 includes a solid-state memory such as a memory card or other package that houses one or more non-volatile read-only memories. The computer-readable medium 522 is a random-access memory or other volatile re-writable memory. Additionally or alternatively, the computer-readable medium 522 includes a magneto-optical or optical medium, such as a disk or tapes or other storage device to capture carrier wave signals such as a signal communicated over a transmission medium. A digital file attachment to an e-mail or other self-contained information archive or set of archives is considered a distribution medium that is a tangible storage medium. Accordingly, the disclosure is considered to include any one or more of a computer-readable medium or a distribution medium and other equivalents and successor media, in which data or instructions are stored.

In an alternative implementation, dedicated hardware implementations, such as application specific integrated circuits, programmable logic arrays, and other hardware devices, is constructed to implement one or more of the methods described herein. Applications that include the apparatus and systems of various implementations broadly include a variety of electronic and computer systems. One or more implementations described herein implement functions using two or more specific interconnected hardware modules or devices with related control and data signals that are communicated between and through the modules, or as portions of an application-specific integrated circuit. Accordingly, the present system encompasses software, firmware, and hardware implementations.

Computer system 500 is connected to the network 105. The network 105 defines one or more networks including wired or wireless networks. The wireless network is a cellular telephone network, an 802.10, 802.16, 802.20, or WiMAX network. Further, such networks include a public network, such as the Internet, a private network, such as an intranet, or combinations thereof, and utilizes a variety of networking protocols now available or later developed including, but not limited to TCP/IP based networking protocols. The network 105 includes wide area networks (WAN), such as the Internet, local area networks (LAN), campus area networks, metropolitan area networks, a direct connection such as through a Universal Serial Bus (USB) port, or any other networks that allows for data communication. The network 105 is configured to couple one computing device to another computing device to enable communication of data between the devices. The network 105 is generally enabled to employ any form of machine-readable media for communicating information from one device to another. The network 105 includes communication methods by which information travels between computing devices. The network 105 is divided into sub-networks. The sub-networks allow access to all of the other components connected thereto or the sub-networks restrict access between the components. The network 105 is regarded as a public or private network connection and includes, for example, a virtual private network or an encryption or other security mechanism employed over the public Internet, or the like.

In accordance with various implementations of the present disclosure, the methods described herein are implemented by software programs executable by a computer system. Further, in an example, non-limited implementation, implementations can include distributed processing, component/object distributed processing, and parallel processing. Alternatively, virtual computer system processing can be constructed to implement one or more of the methods or functionality as described herein.

Although the present specification describes components and functions that are implemented in particular implementations with reference to particular standards and protocols, the disclosure is not limited to such standards and protocols. For example, standards for Internet and other packet switched network transmission (e.g., TCP/IP, UDP/IP, HTML, and HTTP) represent examples of the state of the art. Such standards are periodically superseded by faster or more efficient equivalents having essentially the same functions. Accordingly, replacement standards and protocols having the same or similar functions as those disclosed herein are considered equivalents thereof.

It will be understood that the steps of methods discussed are performed in one embodiment by an appropriate processor (or processors) of a processing (i.e., computer) system executing instructions (computer-readable code) stored in storage. It will also be understood that the disclosure is not limited to any particular implementation or programming technique and that the disclosure is implemented using any appropriate techniques for implementing the functionality described herein. The disclosure is not limited to any particular programming language or operating system.

It should be appreciated that in the above description of example embodiments of the invention, various features of the invention are sometimes grouped together in a single embodiment, figure, or description thereof for the purpose of streamlining the disclosure and aiding in the understanding of one or more of the various inventive aspects. This method of disclosure, however, is not to be interpreted as reflecting an intention that the claimed invention requires more features than are expressly recited in each claim. Rather, as the following claims reflect, inventive aspects lie in less than all features of a single foregoing disclosed embodiment. Thus, the claims following the Detailed Description are hereby expressly incorporated into this Detailed Description, with each claim standing on its own as a separate embodiment of this invention.

Furthermore, while some embodiments described herein include some but not other features included in other embodiments, combinations of features of different embodiments are meant to be within the scope of the invention, and form different embodiments, as would be understood by those skilled in the art. For example, in the following claims, any of the claimed embodiments can be used in any combination.

Furthermore, some of the embodiments are described herein as a method or combination of elements of a method that can be implemented by a processor of a computer system or by other means of carrying out the function. Thus, a processor with the necessary instructions for carrying out such a method or element of a method forms a means for carrying out the method or element of a method. Furthermore, an element described herein of an apparatus embodiment is an example of a means for carrying out the function performed by the element for the purpose of carrying out the invention.

In the description provided herein, numerous specific details are set forth. However, it is understood that embodiments of the invention are practiced without these specific details. In other instances, well-known methods, structures and techniques have not been shown in detail in order not to obscure an understanding of this description.

Thus, while there has been described what are believed to be the preferred embodiments of the invention, those skilled in the art will recognize that other and further modifications are made thereto without departing from the spirit of the invention, and it is intended to claim all such changes and modifications as falling within the scope of the invention. For example, any formulas given above are merely representative of procedures that may be used. Functionality may be added or deleted from the block diagrams and operations may be interchanged among functional blocks. Steps may be added or deleted to methods described within the scope of the present invention.

The above disclosed subject matter is to be considered illustrative, and not restrictive, and the appended claims are intended to cover all such modifications, enhancements, and other implementations, which fall within the true spirit and scope of the present disclosure. Thus, to the maximum extent allowed by law, the scope of the present disclosure is to be determined by the broadest permissible interpretation of the following claims and their equivalents, and shall not be restricted or limited by the foregoing detailed description. While various implementations of the disclosure have been described, it will be apparent to those of ordinary skill in the art that many more implementations and implementations are possible within the scope of the disclosure. Accordingly, the disclosure is not to be restricted except in light of the attached claims and their equivalents.

The present disclosure furthermore relates to the following aspects:

Example 1. A computer-implemented method comprising: accessing, by one or more processors, a plurality of datasets stored in a database; identifying, by the one or more processors, (a) a first subset of the plurality of datasets that each comprise an indicator explicitly representing one or more conditions based on one or more deterministic criteria, and (b) a second subset of the datasets that excludes the indicator and comprises data implicitly representing the one or more conditions based on the one or more deterministic criteria; merging, by the one or more processors, the first subset and the second subset into a first data object; receiving, by the one or more processors and as input via an interactive interface, a user input indicative of a plurality of parameters that correspond to values contained in the first data object; generating, by the one or more processors, a plurality of similarity-based subsets by applying one or more sampling techniques to the first data object across the plurality of parameters, each of the sample similarity-based subsets associated with a deviation measure; generating, by the one or more processors, a second data object comprising one of the plurality of similarity-based subsets associated with a lowest deviation measure; and providing, by the one or more processors, the second data object for display via the interactive interface in response to the user input.

Example 2. The method of Example 1, wherein the second data object further comprises one or more additional similarity-based subsets of the plurality of similarity-based subsets, wherein the one or more additional similarity-based subsets of the second data object are sorted by deviation measure.

Example 3. The method of any of Examples 1-2, wherein the first subset and the second subset each include one or more members, the one or more members being unique to each respective subset.

Example 4. The method of any of Examples 1-3, the method further comprising determining a plurality of representative categories for the first data object by: identifying one or more options associated with each parameter of the plurality of parameters; and identifying one or more unique combinations of options across the plurality of parameters, each unique combination being a representative category of the plurality of representative categories.

Example 5. The method of any of Examples 1-4, further comprising determining the deviation measure for each of the plurality of similarity-based subsets.

Example 6. The method of any of Examples 1-5, wherein the determining a deviation measure for each of the plurality of similarity-based subsets includes: for each representative category of each respective similarity-based subset of the plurality of similarity-based subsets, determining a probability of the representative category being selected from the first data object, determining a probability of the representative category being selected from the respective similarity-based subset, generating a divergence value for the representative category based at least in part on the probability of the representative category being selected from the first data object and/or the probability of the representative category being selected from the respective similarity-based subset, and generating the deviation measure for each respective similarity-based subset based on the divergence values generated for the representative categories of the respective similarity-based subset.

Example 7. The method of example 1, the method further comprising: generating, by the one or more processors, a plurality of member size categories, each member size category associated with a unique number of group members; and assigning, by the one or more processors, each similarity-based subset of the plurality of similarity-based subsets to a member size category based on the number of group members in the respective similarity-based subset.

Example 8. The method of example 7, the method further comprising: determining, for each member size category, a similarity-based subset with the lowest deviation measure.

Example 9. The method of example 8, wherein the second data object further comprises, for each member size category, the similarity-based subset with the lowest deviation measure.

Example 10. The method of example 9, wherein the plurality of parameters includes a threshold deviation measure, and wherein providing the second data object for display via the interactive interface includes: comparing, for each member size category, the deviation measure of the similarity-based subset with the lowest deviation metric against the threshold deviation measure; determining, based on a result of the comparison, satisfactory member size categories; and providing, along with the second data object, a visual indicia of the satisfactory member size categories.

Example 11. A system comprising: one or more storage devices storing instructions; and one or more processors executing the instructions to perform a process including: accessing a plurality of datasets stored in a database; identifying (a) a first subset of the plurality of datasets that each comprise an indicator explicitly representing one or more conditions based on one or more deterministic criteria, and (b) a second subset of the datasets that excludes the indicator and comprises data implicitly representing the one or more conditions based on the one or more deterministic criteria; merging the first subset and the second subset into a first data object; receiving, as input via an interactive interface, a user input indicative of a plurality of parameters that correspond to values contained in the first data object; generating a plurality of similarity-based subsets by applying one or more sampling techniques to the first data object across the plurality of parameters, each of the sample similarity-based subsets associated with a deviation measure; generating a second data object comprising one of the plurality of similarity-based subsets associated with a lowest deviation measure; and providing the second data object for display via the interactive interface in response to the user input.

Example 12. The system of example 11, wherein the second data object further comprises one or more additional similarity-based subsets of the plurality of similarity-based subsets, wherein the one or more additional similarity-based subsets of the second data object are sorted by deviation measure.

Example 13. The system of example 11, wherein the first subset and the second subset each include one or more members, the one or more members being unique to each respective subset.

Example 14. The system of example 11, wherein the process further includes determining a plurality of representative categories for the first data object by: identifying one or more options associated with each parameter of the plurality of parameters; and identifying one or more unique combinations of options across the plurality of parameters, each unique combination being a representative category of the plurality of representative categories.

Example 15. The system of example 14, wherein the process further includes determining a deviation measure for each of the plurality of similarity-based subsets by: for each representative category of each respective similarity-based subsets of the plurality of similarity-based subsets, determining a probability of the representative category being selected from the first data object, determining a probability of the representative category being selected from the respective similarity-based subsets, generating a divergence value for the representative category based at least in part on the probability of the representative category being selected from the first data object and/or the probability of the representative category being selected from the respective similarity-based subset, and generating the deviation measure for each respective similarity-based subset based on the divergence values generated for the representative categories of the respective similarity-based subset.

Example 16. The system of example 11, wherein the process further includes: Generating a plurality of member size categories, each member size category associated with a unique number of group members; and Assigning each similarity-based subset of the plurality of similarity-based subsets to a member size category based on the number of group members in the respective similarity-based subset.

Example 17. The system of example 16, wherein the process further includes: determining, for each member size category, a similarity-based subset with the lowest deviation measure.

Example 18. The system of example 17, wherein the second data object further comprises, for each member size category; the similarity-based subset with the lowest deviation measure.

Example 19. The system of example 18, wherein the plurality of parameters includes a threshold deviation measure, and wherein providing the second data object for display via the interactive interface includes: comparing, for each member size category, the deviation measure of the similarity-based subset with the lowest deviation measure against the threshold deviation measure; determining, based on the comparison, satisfactory member size categories; and providing, along with the second data object, a visual indicia of the satisfactory member size categories.

Example 20. A non-transitory computer-readable medium storing instructions which, when executed by a computer, cause the computer to perform am method comprising: accessing, by one or more processors, a plurality of datasets stored in a database; identifying, by the one or more processors, (a) a first subset of the plurality of datasets that each comprise an indicator explicitly representing one or more conditions based on one or more deterministic criteria, and (b) a second subset of the datasets that excludes the indicator and comprises data implicitly representing the one or more conditions based on the one or more deterministic criteria; merging, by the one or more processors, the first subset and the second subset into a first data object; receiving, by the one or more processors and as input via an interactive interface, a user input indicative of a plurality of parameters that correspond to values contained in the first data object; generating, by the one or more processors, a plurality of similarity-based subsets by applying one or more sampling techniques to the first data object across the plurality of parameters, each of the sample similarity-based subsets associated with a deviation measure; generating, by the one or more processors, a second data object comprising one of the plurality of similarity-based subsets associated with a lowest deviation measure; and providing, by the one or more processors, the second data object for display via the interactive interface in response to the user input.

What is claimed is:

1. A computer-implemented method comprising:
accessing, by one or more processors, a plurality of datasets stored in a database;
identifying, by the one or more processors, (a) a first subset of the plurality of datasets that each comprise an indicator explicitly representing one or more conditions based on one or more deterministic criteria, and (b) a second subset of the datasets that excludes the indicator and comprises data implicitly representing the one or more conditions based on the one or more deterministic criteria;
merging, by the one or more processors, the first subset and the second subset into a first data object;
receiving, by the one or more processors and as input via an interactive interface, a user input indicative of a plurality of parameters that correspond to values contained in the first data object;
generating, by the one or more processors, a plurality of similarity-based subsets by applying one or more sampling techniques to the first data object across the plurality of parameters, each of the sample similarity-based subsets associated with a deviation measure;
generating, by the one or more processors, a second data object comprising one of the plurality of similarity-based subsets associated with a lowest deviation measure; and
providing, by the one or more processors, the second data object for display via the interactive interface in response to the user input.

2. The method of claim 1, wherein the second data object further comprises one or more additional similarity-based subsets of the plurality of similarity-based subsets, wherein the one or more additional similarity-based subsets of the second data object are sorted by deviation measure.

3. The method of claim 1, wherein the first subset and the second subset each include one or more members, the one or more members being unique to each respective subset.

4. The method of claim 1, the method further comprising determining a plurality of representative categories for the first data object by:
identifying one or more options associated with each parameter of the plurality of parameters; and
identifying one or more unique combinations of options across the plurality of parameters, each unique combination being a representative category of the plurality of representative categories.

5. The method of claim 4, further comprising determining the deviation measure for each of the plurality of similarity-based subsets.

6. The method of claim 5, wherein the determining a deviation measure for each of the plurality of similarity-based subsets includes:
for each representative category of each respective similarity-based subset of the plurality of similarity-based subsets,
determining a probability of the representative category being selected from the first data object,
determining a probability of the representative category being selected from the respective similarity-based subset,
generating a divergence value for the representative category based at least in part on the probability of the representative category being selected from the first data object and/or the probability of the representative category being selected from the respective similarity-based subset, and
generating the deviation measure for each respective similarity-based subset based on the divergence values generated for the representative categories of the respective similarity-based subset.

7. The method of claim 1, the method further comprising:
generating, by the one or more processors, a plurality of member size categories, each member size category associated with a unique number of group members; and
assigning, by the one or more processors, each similarity-based subset of the plurality of similarity-based subsets to a member size category based on the number of group members in the respective similarity-based subset.

8. The method of claim 7, the method further comprising: determining, for each member size category, a similarity-based subset with the lowest deviation measure.

9. The method of claim 8, wherein the second data object further comprises, for each member size category, the similarity-based subset with the lowest deviation measure.

10. The method of claim 9, wherein the plurality of parameters includes a threshold deviation measure, and wherein providing the second data object for display via the interactive interface includes:
comparing, for each member size category, the deviation measure of the similarity-based subset with the lowest deviation metric against the threshold deviation measure;
determining, based on a result of the comparison, satisfactory member size categories; and
providing, along with the second data object, a visual indicia of the satisfactory member size categories.

11. A system comprising:
one or more storage devices storing instructions; and
one or more processors executing the instructions to perform a process including:
accessing a plurality of datasets stored in a database;
identifying (a) a first subset of the plurality of datasets that each comprise an indicator explicitly representing one or more conditions based on one or more deterministic criteria, and (b) a second subset of the datasets that excludes the indicator and comprises data implicitly representing the one or more conditions based on the one or more deterministic criteria;
merging the first subset and the second subset into a first data object;
receiving, as input via an interactive interface, a user input indicative of a plurality of parameters that correspond to values contained in the first data object;
generating a plurality of similarity-based subsets by applying one or more sampling techniques to the first data object across the plurality of parameters, each of the sample similarity-based subsets associated with a deviation measure;
generating a second data object comprising one of the plurality of similarity-based subsets associated with a lowest deviation measure; and
providing the second data object for display via the interactive interface in response to the user input.

12. The system of claim 11, wherein the second data object further comprises one or more additional similarity-based subsets of the plurality of similarity-based subsets, wherein the one or more additional similarity-based subsets of the second data object are sorted by deviation measure.

13. The system of claim 11, wherein the first subset and the second subset each include one or more members, the one or more members being unique to each respective subset.

14. The system of claim 11, wherein the process further includes determining a plurality of representative categories for the first data object by:
identifying one or more options associated with each parameter of the plurality of parameters; and
identifying one or more unique combinations of options across the plurality of parameters, each unique combination being a representative category of the plurality of representative categories.

15. The system of claim 14, wherein the process further includes determining a deviation measure for each of the plurality of similarity-based subsets by:
for each representative category of each respective similarity-based subsets of the plurality of similarity-based subsets,
determining a probability of the representative category being selected from the first data object,
determining a probability of the representative category being selected from the respective similarity-based subsets,
generating a divergence value for the representative category based at least in part on the probability of the representative category being selected from the first data object and/or the probability of the representative category being selected from the respective similarity-based subset, and
generating the deviation measure for each respective similarity-based subset based on the divergence values generated for the representative categories of the respective similarity-based subset.

16. The system of claim 11, wherein the process further includes:
Generating a plurality of member size categories, each member size category associated with a unique number of group members; and
Assigning each similarity-based subset of the plurality of similarity-based subsets to a member size category based on the number of group members in the respective similarity-based subset.

17. The system of claim 16, wherein the process further includes: determining, for each member size category, a similarity-based subset with the lowest deviation measure.

18. The system of claim 17, wherein the second data object further comprises, for each member size category; the similarity-based subset with the lowest deviation measure.

19. The system of claim 18, wherein the plurality of parameters includes a threshold deviation measure, and wherein providing the second data object for display via the interactive interface includes:
comparing, for each member size category, the deviation measure of the similarity-based subset with the lowest deviation measure against the threshold deviation measure;
determining, based on the comparison, satisfactory member size categories; and
providing, along with the second data object, a visual indicia of the satisfactory member size categories.

20. A non-transitory computer-readable medium storing instructions which, when executed by a computer, cause the computer to perform a method comprising:
accessing, by one or more processors, a plurality of datasets stored in a database;
identifying, by the one or more processors, (a) a first subset of the plurality of datasets that each comprise an indicator explicitly representing one or more conditions based on one or more deterministic criteria, and (b) a second subset of the datasets that excludes the indicator and comprises data implicitly representing the one or more conditions based on the one or more deterministic criteria;
merging, by the one or more processors, the first subset and the second subset into a first data object;
receiving, by the one or more processors and as input via an interactive interface, a user input indicative of a plurality of parameters that correspond to values contained in the first data object;
generating, by the one or more processors, a plurality of similarity-based subsets by applying one or more sampling techniques to the first data object across the plurality of parameters, each of the sample similarity-based subsets associated with a deviation measure;
generating, by the one or more processors, a second data object comprising one of the plurality of similarity-based subsets associated with a lowest deviation measure; and
providing, by the one or more processors, the second data object for display via the interactive interface in response to the user input.
